(19) United States
(12) Patent Application Publication
(10) Pub. No.: US 2006/0009694 A1
(43) Pub. Date: Jan. 12, 2006

(54) Methods of attenuating internal radiation exposure

(76) Inventors: David K. Yousefzadeh, Skokie, IL (US); Matthew Ward, Chicago, IL (US); Chester Reft, Park Forest, IL (US); Charles Pelizzari, Western Springs, IL (US)

Correspondence Address: BRINKS HOFER GILSON & LIONE, P.O. BOX 10395, CHICAGO, IL, 60610, US

(21) Appl. No.: 11/131,433

(22) Filed: May 17, 2005

Related U.S. Application Data
(60) Provisional application No. 60/572,581, filed on May 17, 2004.

(51) Int. Cl. A61B 6/00

(52) U.S. Cl. 600/431; 600/436

(57) ABSTRACT

Various radiation-attenuating agents can be administered within a patient body cavity in combination with therapeutic irradiation of the patient body cavity, such as during radiographic diagnostic imaging procedures, to attenuate the radiation incident on certain portions of the patient body cavity believed to be susceptible to harm from the incident radiation. A variety of methods are provided, including methods for detecting clinical conditions such as pulmonary embolus in a pregnant woman while mitigating uterine radiation exposure.

This application claims priority to provisional patent application Ser. No. 60/572,581, filed May 17, 2004.

### TECHNICAL FIELD

The present invention relates, in general, to methods of attenuating radiation exposure within a patient body cavity so as to minimize adverse effects of the radiation. The methods can be used in radiographic diagnostic imaging of a patient body cavity. For example, the methods can be used in chest computed tomography of pregnant patients suspected of having pulmonary embolus.

### BACKGROUND

Irradiation of portions of a patient body cavity is routinely practiced in medical treatment, for example as part of a variety of diagnostic imaging procedures. For example, diagnostic imaging of a patient body cavity may include irradiation using X-rays as part of procedures such as computed tomography (hereinafter CT) scans. Diagnostic imaging is particularly useful in diagnosing and preventing a variety of medical conditions.

However, irradiation of certain portions of a patient body cavity carries certain medical risks. Therefore, while diagnostic imaging using ionizing radiation such as X-rays may be particularly beneficial to identify and treat deleterious medical conditions, such diagnostic imaging techniques may simultaneously present increased potential risk to the patient. Certain medical conditions that can be beneficially diagnosed using medical procedures comprise the step of irradiating a portion of a patient body cavity. However, exposure to the radiation can undesirably subject the patient to medical risk from deleterious radiation effects.

In certain circumstances, a patient can be at greater risk for a condition that can be diagnosed by a medical procedure comprising the step of irradiation of a portion of the patient body cavity, while the patient can also be at a greater risk from the potentially undesirable medical effects of exposure to the same irradiation. The detection of pulmonary embolism (PE) in pregnant women provides one illustrative example where the patient is at increased risk for pulmonary embolism during pregnancy, and simultaneously increasingly susceptible to medical risks associated with detection of PE using X-rays. An embolism is a blood clot, a lump of coagulated blood that breaks off from the wall of a vein and travels with the blood to other organs in the body. Pulmonary embolism, also referred to as a lung embolus, occurs when a blood vessel supplying the lung becomes clogged up by a blood clot. The American Heart Association statistics indicate that about 600,000 Americans develop pulmonary embolism each year, resulting in about 60,000 deaths per year. PE is now diagnosed best by a CT scan. There is a five to six-fold increased risk for PE in pregnancy, which continues to be the leading non-obstetric cause of death during pregnancy or in the peripartum period. However, exposure to X-rays poses increased risk to the well-being of the fetus. The fetal radiation dose with single-detector CT has been estimated to be between 0.3 and 200 mrad (Damilakis J et al., 2000, Invest Radiol 35: 527-533). For a variety of reasons such as poor contrast opacification, motion artifact, and other technical factors, about 5%-10% of CT scans are non-diagnostic, and patients may either need a repeat study or may undergo additional examinations, such as lung scintigraphy or pulmonary angiography. Even though multi-detector CT (MDCT) may be a preferred imaging tool, the MDCT technology provides a tradeoff for safety in the quest for improving diagnostic accuracy.

During radiographic X-ray imaging, external lead shielding is often used to shield portions of the body cavity believed to be at risk for damage from irradiation, such as the gonads or uterus. However, the external shielding does not block the scattered radiation during CT examination, which is transmitted transdiaphragmatically toward the abdomen and its contents including the fetus during the pregnancy. In addition, the exact location and size of various organs within the body cavity vary from patient to patient, leading to a risk that external shielding may be misplaced. Also, radiographic technicians may forget to place the external shielding on a patient. Furthermore, external shielding is largely planar and may not be suitable for medical procedures where irradiation is directed at the body cavity from various angles, such as in a CT scan.

Therefore, there is a need for methods of attenuating the scattered radiation within the body cavity that permit imaging of the body cavity while attenuating the radiation exposure to other parts of the body cavity that are believed to be more susceptible to damage from the irradiation. It is particularly important to minimize the radiation dose absorbed by the fetus who is more susceptible than children and adults.

### BRIEF SUMMARY

Methods of attenuating scatter radiation within the body cavity comprising the administration of a radiation-attenuating agent within at least a portion of a patient body cavity are provided. Methods of irradiating a patient comprising the step of administering a radiation-attenuating agent before or during the radiation are also provided herein. The irradiation is preferably performed as part of a therapeutic or diagnostic medical procedure, such as diagnostic medical imaging using X-ray radiation.

The radiation-attenuating agent is preferably administered to a first portion of a patient body cavity. The first portion of the patient body cavity can be selected to attenuate radiation incident upon a second portion of the patient body cavity during the irradiation. Preferably, the first portion is the alimentary canal (tract) or a portion thereof. The second portion of the patient body can be any part of the anatomy believed to be susceptible to increased risk of harm by irradiation, such as the gonads or uterus. Also preferably, the administered radiation-attenuating agent is maintained within the first portion of the patient body cavity during irradiation of the patient. The irradiation of the body cavity, or any portion thereof, is conducted for a therapeutically effective manner, such as to produce a diagnostic image or treat a tumor site. Any suitable radiation can be employed, such as X-rays, optionally in combination with a medical diagnostic imaging technique. Any suitable radiation-attenuating agent capable of attenuating the radiation incident upon a body cavity can be used. Preferably, the radiation-attenuating agent is a radiographic contrast agent, such as a barium salt. Most preferably, the radiation-attenuating agent is barium sulfate.

The method of irradiating a patient can comprise the steps of administering a radiation-attenuating agent within a first portion of a patient body cavity, and irradiating a second portion of the patient body cavity with a therapeutically effective source of radiation. The second portion of the patient body cavity may be the same as, different than, or overlap with the first portion of the patient body cavity. For example, the first patient body cavity portion may be a portion of the alimentary canal, and the second patient body cavity portion may be a portion of the thorax of the patient. Preferably, the administered radiation-attenuating agent is positioned to attenuate at least a portion of the radiation incident upon the first portion of the patient body cavity. A method of irradiating a patient can optionally further comprise the steps of detecting radiation passing through the second portion of the patient body cavity; or providing a diagnostic image showing at least the second portion of the patient body cavity.

The method of irradiating a patient can be performed as part of a method of diagnostic imaging. Methods of diagnostic imaging can comprise the steps of: administering a radiation-attenuating agent within a first portion of a patient body cavity, where the administered radiation-attenuating agent is positioned to attenuate radiation incident upon a second portion of the patient body cavity during the irradiation of the first portion of the patient body cavity; and the step of irradiating a third portion of the patient body cavity with X-ray radiation, where the third portion of the patient body cavity comprises at least a portion of the first portion of the patient body cavity. The first, second and third portions of the patient body cavity may be the same as, different than, or overlap with one another. For example, the first patient body cavity portion may be a portion of the alimentary canal, the second patient body cavity portion may be a gonad or uterus, and the third patient body cavity portion may be a portion of the thorax.

The method of irradiating a patient can also be performed as part of a method of detecting a clinical condition, such as a pulmonary embolism. Methods of detecting clinical conditions may comprise the steps of: administering a radiation-attenuating agent comprising barium within a first portion of a patient body cavity; and performing a diagnostic imaging procedure of the patient body cavity, where the diagnostic imaging procedure comprises the step of irradiating the first portion of the patient body cavity and a second portion of the patient body cavity with X-ray radiation; where the administered radiation-attenuating agent attenuates radiation incident upon a gonad or the uterus of the patient during the diagnostic imaging procedure.

### BRIEF DESCRIPTION OF THE DRAWINGS

FIG. 1 shows relative attenuation profiles as a function of different concentrations of barium-containing radiation-attenuating agents used in near and far fields, and extrapolated for 1 and 2 cm thick slabs containing different concentrations of barium sulfate.

### DETAILED DESCRIPTION OF THE PRESENTLY PREFERRED EMBODIMENTS

It is to be understood that this invention is not limited to the particular methodology, protocols, patients, or reagents described, and as such may vary. It is also to be understood that the terminology used herein is for the purpose of describing particular embodiments only, and is not intended to limit the scope of the present invention, which is preferably limited only by the claims.

As used herein, recitation of the term “irradiation” or “radiation” refers to X-rays outside the visible spectrum that are contacted with a patient for diagnostic or therapeutic purposes. X-rays are one particularly preferred form of radiation. For example, patients undergoing CT scans are irradiated with X-rays. During the CT scanning process, the patients are exposed to X-ray radiation. A pregnant patient undergoing diagnosis or treatment is not only exposed to radiation herself; the fetus of a pregnant person is exposed to radiation as well.

A variety of radiation-attenuating agents may be administered to a patient prior to or during irradiation of a portion of a patient body cavity. Irradiation of the body cavity can be performed for any suitable diagnostic or therapeutic purpose, including diagnostic imaging or treatment of cancer. The radiation-attenuating agent is preferably administered within a portion of the patient body cavity so as to attenuate the radiation incident, scattered on another portion of the patient body cavity.

Preferably, the radiation-attenuating agent shields a portion of the patient body cavity from levels of radiation that are believed to be harmful to the shielded body cavity portion. Irradiation of a first portion of the patient body cavity may result in scattered radiation that could harm a second portion of the patient body cavity. Even though the second portion of the patient body cavity may be externally shielded, the scattered radiation from the first, irradiated portion of the body cavity may be in the direction of the second patient body cavity.

As used herein, recitation of the terms “shield” or “shielding,” refer to the attenuation of levels of scattered radiation that are believed to be harmful to a portion of a body cavity. Scattered radiation typically occurs when one portion of a patient's body is irradiated and that radiation is deflected into a second portion of the body not initially irradiated. Preferably, administration of a radiation-attenuating agent in a first portion of a patient body cavity shields a second portion of the patient body cavity from scattered radiation. For example, the first portion of the patient body cavity can be a portion of the intestine and the second portion of the patient body cavity can be the uterus of a pregnant patient.

Radiation-Attenuating Agents

A radiation-attenuating agent can be any biocompatible composition that attenuates radiation, and preferably an agent that attenuates X-ray radiation. Any suitable radiation-attenuating agent can be used that provides a desirable level of radiation attenuation. The radiation-attenuating agent can be a radiographic contrast agent that absorbs or attenuates radiation, such as X-ray radiation used in a diagnostic imaging procedure. For some medical imaging procedures, a contrast agent is introduced to a portion of a patient body cavity to image the portion of a patient body cavity containing the contrast agent. X-ray visualization of organs containing soft tissue, such as the gastrointestinal (hereinafter GI) tract, often includes introducing a contrast agent, which attenuates X-ray radiation to the organ being imaged. D. P. Swanson et al in “Pharmaceuticals In Medical Imaging,” 1990, MacMillan Publishing Company, provides a background in medical imaging utilizing contrast agents and compositions. For example, U.S. Pat. No. 5,405,600 (Illig et al.), incorporated herein by reference, discloses certain X-ray contrast compositions containing film-forming materials that can be used as radiation-attenuating agents.

Preferably, the radiation-attenuating agent comprises barium, such as a barium salt, although other radiation-attenuating agents can also be used. One preferred radiation-attenuating agent is barium sulfate, which can be administered in any suitable manner, such as an oral suspension. Barium sulfate is preferable for administration to the alimentary tract (canal) of a patient, in part because it is not absorbable from the GI tract following oral administration, and because of speedy excretion from the body. Accordingly, administration of a radiation-attenuating agent is preferred for administration to the alimentary canal. Barium sulfate has the empirical formula of BaSO₄ and is a white, radiopaque, crystalline powder that is essentially insoluble in water. However, good results are obtainable with other inorganic, essentially water-insoluble salts of barium including barium hexaboride, barium chromate, barium fluorogallate, barium tri-ortho phosphate, barium metasilicate, barium titanate, barium zirconate and zirconium oxide. Other examples of radiation-attenuating agents comprising barium include, for example, those disclosed in U.S. Pat. Nos. 2,659,690; 2,680,089; 3,216,900; 3,235,462; 4,038,379 and 4,120,946, which are incorporated herein by reference. U.S. Pat. No. 4,120,946, also incorporated by reference, discloses a pharmaceutical composition for barium opacification of the digestive tract, comprising colloidal barium sulfate and a polyacrylamide in an aqueous vehicle. The polyacrylamide forms a viscous solution at low concentration, which makes it possible to maintain the barium sulfate in suspension and at the same time permit good adherence of the preparation to the walls of the organ that it is desired to X-ray.

Radiographic contrast agents comprising iodinated organic compounds can also be used as a radiation-attenuating agent, as the iodine atom is an effective X-ray absorber. Iodinated organic compounds absorb X-rays and produce a so-called photoelectric effect which is a large magnification in contrast caused by the photons stopped in the iodine-containing medium. The magnification of contrast is believed to exceed the level that would be expected from relative changes in density. As a result of this magnification, relatively low concentrations of the iodinated organic radiographic contrast agents can be utilized as radiation-attenuating agents. Examples of iodinated radiation-attenuating agents include, for example, those disclosed in U.S. Pat. Nos. 2,786,055; 3,795,698; 3,360,436; 3,574,718, 3,733,397; 4,735,795; 5,047,228; and 5,468,466, which are incorporated herein by reference.

Radiation-attenuating agents, such as a radiographic contrast agent, can also comprise various polymer compositions designed to enhance attachment of the radiation-attenuating agent to portions of a patient body cavity. U.S. Pat. No. 4,069,306, incorporated by reference, discloses an X-ray contrast preparation adapted to adhere to the walls of body cavities. The preparation comprises a finely divided water-insoluble inorganic X-ray contrast agent and minute particles of a hydrophilic polymer, which is insoluble in water but is water-swellable. The body cavity is supplied with such preparation suspended in water. The X-ray contrast agent is present in admixture with and/or enclosed in and/or adhered to said minute polymer particles. U.S. Pat. No. 5,019,370, incorporated by reference, discloses a biodegradable radiographic contrast medium comprising biodegradable polymeric spheres, which carry a radiographically opaque element, such as iodine, bromine, samarium and erbium. The contrast medium is provided either in a dry or liquid state and may be administered intravenously, orally and intra-arterially. Japanese Pat. Application No. 55-127322, incorporated by reference, discloses X-ray contrast compositions containing barium sulfate and a polymeric substance selected from carboxymethyl cellulose salts, propylene glycol alginate, cellulose sulfate polyacrylate, pectin, and tragacanth gum. The polymeric substance is used to increase the viscosity of the compositions.

Therapeutic Compositions Comprising a Radiation-Attenuating Agent

The radiation-attenuating agent can be administered as part of a composition in any pharmaceutically biocompatible form. A variety of suitable excipients and vehicles are suitable for administering the composition comprising the radiation-attenuating agent. Preferably, a composition comprising the radiation-attenuating agent is a radiographic contrast composition. While compositions comprising a radiation-attenuating agent are discussed below with respect to a radiation-attenuating agent comprising barium for attenuation of X-ray radiation, the disclosure demonstrates that any suitable radiation-attenuating agent can be formulated into suitable compositions for administration within a portion of a patient body cavity. The exact formulation selected can depend on which portion of the patient body cavity the radiation-attenuating agent is being administered to, the desired level and duration of radiation attenuation, and the type of radiation used.

In one embodiment, a barium salt radiation-attenuating agent is formulated for administration using physiologically acceptable carriers or excipients in a manner within the skill of the art. The barium salt, with the addition of desirable pharmaceutically acceptable aids (such as surfactants and emulsifiers) and excipients, may be suspended in an aqueous medium resulting in a dispersion, suspension or colloid. Liquid compositions used for practicing the method of the present invention comprise the following pharmaceutically acceptable components based on % w/w: barium salt 5-95; excipient 0-20; aids (surfactants/emulsifiers) 0.01-20; water q.s. to 100. Artifacts in the diagnosis media can occur when the concentration of barium salt is high. Those of skill in the art can recognize such artifacts when present. Such artifacts can be minimized by lowering the concentration of barium salt.

Various formulations of barium sulfate suspensions are known in the art (e.g., HD 85® Barium Sulfate suspension, Malinckrodt Inc.). These suspensions are utilized for purposes other than this invention. In a preferred embodiment, in addition to the barium salt, liquid compositions used for practicing the method of the present invention may contain suspending and dispersing agents, simethicone, citric acid, flavoring, potassium sorbate, sodium citrate, saccharin sodium or other artificial sweetener and water.

Solid barium sulfate is commercially available in a broad range of particle sizes. Commercial products generally contain material ranging from 400 nanometers to 10 μm and have a mean average diameter of 2 μm. These particles may be individual particles or aggregates of smaller particles. The commercially available barium salts are formulated into compounds useful in the present invention by various methods known in the art, such as solubilization, wet milling, microfluidization, fluid air jet milling, high-shear mixing and controlled precipitation.

The aqueous vehicle for the barium salt suspension used to practice the invention may contain a combination of stabilizers and additional excipients. Additional ingredients known to these skilled in the art may also be included. Ingredients routinely employed in pharmaceutical suspensions include sweetening and flavoring agents (such as saccharine sodium), colors, lakes, dyes, antifoam agents (such as simethicone), preservatives (such as parabens or benzoic and sorbic acids) and viscosity enhancers. The order of addition prior to mixing is unimportant, provided that the individual barium particles are ultimately wetted. The contents are then subjected to a mixing or blending process sufficient to disperse the materials uniformly throughout the product.

Excipients advantageously used in the formulations include viscosity mediating and stabilizing agents, such as microcrystalline cellulose, ethylcellulose, hydroxypropyl methylcellulose and gum arabic. Physiologically acceptable substances may also be included, such as sodium citrate, sodium chloride, therapeutic substances, antacid substances and flavoring agents. The inclusion of antimicrobial/antiseptic agents such as methyl parahydroxybenzoate, ethyl parahydroxybenzoate, propyl parahydroxy-benzoate, benzoic acid, benzyl alcohol, phenol, sodium benzoate, EDTA or sorbic acid may also be desirable in some formulations.

Surfactants or emulsifiers can be used alone or in combination with other emulsifying agents and surfactants. For example, Dow Corning Medical Antifoam AF, which is a composition of 30% w/v polydimethylsiloxane and silica aerogel, 14% w/v stearate emulsifiers and 0.075% w/v sorbic acid, the balance being water, may be used by itself. The amount of such surfactants may be in the range of from 0.01 to 20% w/w of the aqueous formulations, although the amount, in general, is kept as low as possible, preferably in the range of 0.05 to 5% w/w. The surface active agents may be cationic, anionic, nonionic, zwitterionic or a mixture of two or more of these agents. Suitable cationic surfactants include cetyl trimethyl ammonium bromide. Suitable anionic agents include sodium lauryl sulphate, sodium heptadecyl sulphate, alkyl benzenesulphonic acids and salts thereof, sodium butylnapthalene sulfonate, and sulphosuccinates. Zwitterionic surface active agents are substances that when dissolved in water they behave as diprotic acids and, as they ionize, they behave both as a weak base and a weak acid.

Alternatively, the barium salt and the pharmaceutically acceptable carrier may be formulated into a solid form, such as tablets or capsules. Solid compositions used for practicing the method of the present invention shall contain, instead of surfactants/emulsifiers and water used in the liquid compositions, bulking agents and other pharmaceutically acceptable ingredients advantageously employed to render the compositions palatable. When the barium salt is formulated as a tablet, the bulking agent should have good compression characteristics. Suitable bulking agents are well known in the art and include a sweetener such as sugars, for example sucrose, and polyhydric alcohols, for example mannitol, sorbitol and xylitol, and mixtures thereof. When formulated as a tablet, it is preferable to incorporate in the composition one or more tablet lubricating agents, such as stearic acid, magnesium stearate and talc. The skilled formulator can readily determine the amount of the tablet lubricating agents as well as any other ingredients required to easily prepare the solid compositions. The solid compositions may have incorporated therein optional pharmaceutically acceptable ingredients in order to impart thereto additional desirable properties, such as flavorants and colorants.

Solid compositions used for practicing the method of the present invention comprise the following pharmaceutically acceptable components based on % w/w: barium salt 5-95; bulking agent/lubricant/flavorant/colorant q.s. to 100.

The stabilized barium salt is formulated for administration using physiologically acceptable carriers or excipients in a manner within the skill of the art. The barium salt and the combination of stabilizers, with the addition of pharmaceutically acceptable aids and excipients are suspended in an aqueous medium resulting in a dispersion or suspension.

The primary stabilizers are nonionic, and are believed to possess adhesive properties. Nonionic surface stabilizers useful in the invention include: cellulose-based polymers such as methyl cellulose, hydroxypropyl cellulose and hydroxypropyl methyl cellulose (such as Methocel K4M—sold by the Dow Chemical Company), carboxylic esters (such as sorbitan esters) and ethoxylated derivatives, carboxylic amides, ethoxylated alkylphenols and ethoxylated alcohols, ethylene oxide/propylene oxide copolymers (such as poloxamer 407, 188, 237 and 338, and other poloxamer polymers such as those sold by BASF under the trademark Pluronic), polyvinyl pyrrolidone and polyvinyl alcohol. Preferred are hydroxypropyl methyl cellulose and ethylene oxide/propylene oxide tri-block copolymers. The compositions used for practicing the method of the present invention comprise nonionic stabilizer in the range of 0.25-25% w/v, preferably 1.0-10% w/v, most preferably 4.0-6.0% w/v.

The secondary stabilizer is an anionic stabilizer including zwitterionic stabilizers, which possess a net negative charge at physiologically relevant pH. Suitable anionic stabilizers include sodium alkyl sulfates (such as sodium dodecyl sulfate) and related salts, alkyl benzenesulfonic acids and salts thereof, butyl naphthalene sulfonates and sulfosuccinates. The compositions used for practicing the method of the present invention comprise an anionic stabilizer in the range of 0.001-5% w/v, preferably 0.01-0.5% w/v, most preferably 0.05-0.2% w/v.

Alternatively, the formulations may be prepared by methods other than direct incorporation (such as controlled precipitation of, for example, barium sulfate from soluble barium salts).

Radiation-Attenuating Agent Dosages

The dosages of the radiation-attenuating agent used according to the methods of the present invention can vary according to the precise nature of the irradiation method and the tissues and organs targeted during the procedure. Preferably, however, the dosage should be kept as low as is consistent with achieving attenuation of undesirable levels of radiation. Preferably, the concentration of a barium salt radiation-attenuating agent should be in the range of from about 1% w/v to about 99% w/v of the formulation, preferably from about 10% w/v to about 60% w/v and most preferably of from about 15% w/v to about 40% w/v.

For CT scanning with typical formulations of compositions comprising a barium salt radiation-attenuating agent, the dosage of barium (Ba) is preferably in the range of from about 1 to about 800 mg Ba/kg body weight, preferably in the range of from about 15 to about 250 mg Ba/kg body weight, and most preferably in the range of from about 35 to about 90 mg Ba/kg body weight. These dosages can provide for attenuation of the effect of radiation on a fetus in a pregnant woman, or for attenuation of the effect of radiation on internal tissues and organs.

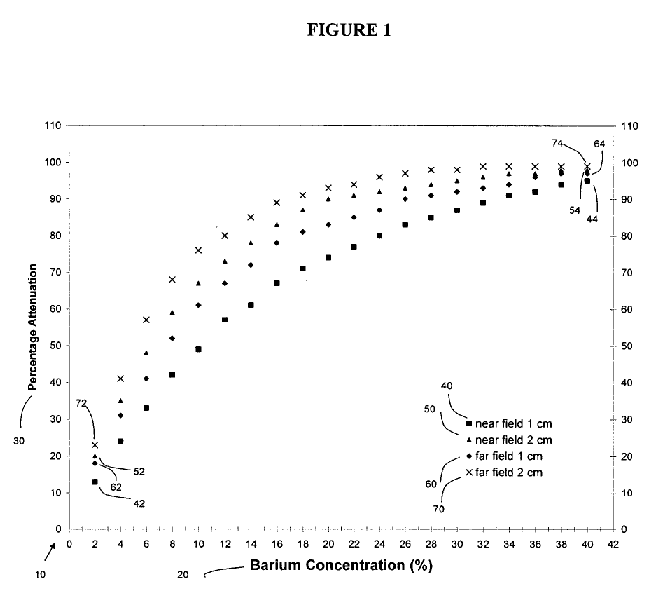

The amount of attenuation of the radiation can depend on the type and dose of radiation-attenuating agent selected. For example, the dose of radiation-attenuating agent can be determined or estimated based on radiation detection methods known in the art. As shown in FIG. 1 and as described in the examples below, the level of radiation attenuation as a function of radiation-attenuating agent concentration is described for 2% concentration of barium sulfate (see Table I below). However, the radiation intensity for various radiation-attenuating agents can be determined by properties of radiation-attenuating agents known in the art, or from the experimental method described in Example 1 below.

FIG. 1 shows a graph 10, which shows extrapolated composite relative attenuation of radiation 30 as a function of different (0-40%) concentrations of barium in a radiation-attenuating agent 20 described in Example 1 below. The graph 10 shows the extrapolated percentage attenuation of radiation 30 for one cm (40, 60) and two cm (50, 70) thick slabs containing solution of barium sulfate, and used as a shield in the near and the far fields. The near field (z=0) corresponds to the L2 plane, and the far field (z=10 cm) corresponds to the L5 plane. These two fields (near and far) represent the approximate positions of the dome of the uterus in last and first trimesters of pregnancy respectively.

The symbols indicate percentage attenuation data: ▪, attenuation in near field using a 1 cm thick slab 40; ▴, attenuation in near field using a 2 cm thick slab 50; ♦, attenuation in far field using a 1 cm thick slab 60; x, attenuation in far field using a 2 cm thick slab 70. Over the range of barium concentrations 20 tested (0-40%), the minima and the maxima for percentage attenuation are as follows: for attenuation in near field using a 1 cm thick slab 40, the minimum 42 is approximately 13% attenuation, and the maximum 44 is approximately 95% attenuation; for attenuation in near field using a 2 cm thick slab 50, the minimum 52 is approximately 20% attenuation, and the maximum 54 is approximately 99% attenuation; for attenuation in far field using a 1 cm thick slab 60, the minimum 62 is approximately 18% attenuation, and the maximum 64 is approximately 99% attenuation; for attenuation in far field using a 2 cm thick slab 70, the minimum 72 is approximately 23% attenuation, and the maximum 74 is approximately 99% attenuation.

As illustrated by the data in FIG. 1, a variety of concentrations and volume of the radiation-attenuating agent may be used to provide a beneficial shielding effect. More specifically, FIG. 1 illustrates a variety of barium-containing radiation-attenuating agent volumes and concentrations. While a radiation-attenuating agent comprising a 40% barium suspension is used to collect some of the data in FIG. 1, other barium concentrations may be used. Even a 5-10% barium concentration can save the fetus 45%-69% of radiation exposure (FIG. 1). At a 30% concentration, the attenuation curves plateau irrespective of the volume of barium consumed, both in the near and in the far field. Beyond 30%, further radiation savings is preferably only slight. Therefore, the effectiveness of 30% or more of barium suspension approaches the attenuating effect of a 1 mm-thick lead sheet. It is noteworthy that the attenuating effect of 40% barium suspension during the actual experiment for 1 cm and 2 cm slabs was the same as the values obtained by the extrapolation method (90%-98% vs. 91%-99%).

Administration of a Radiation-Attenuating Agent

Compositions comprising the radiation-attenuating agent can be administered in any suitable manner, and in any suitable formulation. Preferably, the radiation attenuation agent is administered to a first portion of a patient body cavity at any suitable time before or during irradiation of a second portion of the body cavity.

Preferably, the radiation-attenuating agent is administered to at least a portion of the alimentary canal of a patient body cavity, including any portion of the gastrointestinal tract. The alimentary canal (tract) includes the esophagus, stomach, small intestine and the large intestine. Characteristics of preferred radiation-attenuating agents for administration to the alimentary canal of a patient include: desirable toxicological profile; the ability to fill the entire bowel/lumen; palatability and nonirritation to the intestinal mucosa; and passing through the GI tract without producing artifacts or stimulating vigorous intestinal peristalsis. Administration of the radiation-attenuating agent to the alimentary canal can be performed by suitable methods know in the art, including, for example, by oral administration or by an enema. Preferably, barium salt is orally administered about five minutes to about one hour prior to irradiation of the patient.

In one embodiment, the radiation-attenuating agent comprising a barium salt is administered orally to a patient. For example, the barium salt may be administered orally to a patient for radiological examination of internal organs. These internal organs may or may not include the gastrointestinal tract. The oral administration of barium salt can provide for attenuating the effects of radiation during irradiation of the patient and his/her internal tissues and organs, or a fetus when the patient is pregnant.

Methods Comprising Administration of Radiation-Attenuating Agents

Radiation-attenuating agents can be administered in a variety of medical procedures to diagnose or treat certain medical conditions, and may be used in combination with existing radiographic diagnostic imaging methods, particularly imaging methods employing X-ray radiation.

In one preferred embodiment, a method of detecting a clinical condition such as pulmonary embolism is provided. In one embodiment, a method of diagnostic imaging is provided, which can comprise the steps of: administering a radiation-attenuating agent within a first portion of a patient body cavity, where the administered radiation-attenuating agent is positioned to attenuate radiation incident upon a second portion of the patient body cavity; and irradiating a third portion of the patient body cavity with X-ray radiation, where the third portion of the patient body cavity comprises at least a portion of the first portion of the patient body cavity. For example, in one embodiment, the radiation-attenuating agent comprises barium that is administered to a portion of the intestine prior to performing an X-ray of the thoracic cavity of a pregnant patient, such that the radiation-attenuating agent shields the uterus of the patient from the X-ray radiation.

When conducting a diagnostic image of a pregnant patient using X-rays, there is a need to balance the need for studies using ionizing radiation with the potential risk to the fetus and the likelihood of cancer caused by pediatric computed tomography (CT). The administration of a radiation-attenuating agent within a portion of the patient body cavity can mitigate the risk to the pregnancy associated with the use of X-rays in the diagnostic procedure. Accordingly, methods of detecting a clinical condition are also provided that comprises the steps of: administering a radiation-attenuating agent comprising barium within a first portion of a patient body cavity; and performing a diagnostic imaging procedure of the patient body cavity comprising the step of irradiating the first portion of the patient body cavity and a second portion of the patient body cavity with X-ray radiation; where the administered radiation-attenuating agent attenuates radiation incident upon a gonad or the uterus of the patient during the diagnostic imaging procedure. Optionally, the method of detecting a clinical condition can further comprise the step of collecting a diagnostic image of the second portion of the body cavity, where the clinical condition is a pulmonary embolism; the diagnostic imaging procedure is a CT scan.

Methods of treatment comprising administration radiation-attenuating agent can be administered in combination with one or more known diagnostic imaging techniques. For example, for diagnosing acute PE, diagnostic techniques include: D-dimer assay (Schoepf U J, Costello P, 2004, Radiology 230: 329-337); lower-extremity ultrasound for DVT, which may indicate risk of PE and justify anticoagulant treatment; contrast enhanced magnetic resonance angiography (MRA) (Schoepf U J et al., 2004, Circulation 109: 2160-2167); nuclear medicine (NM) test, used less frequently because its results are indeterminate in up to 73% of cases (Blachere H et al., 2000, AJR 174:1041-1047); Pulmonary angiography (Stein P D et al., 1999, Radiology 210: 689-691); and Spiral CT angiography, which is now preferred for diagnosing PE by direct visualization of the thrombus. Up to ⅔ of patients with suspected PE have disorders that simulate PE, including aortic dissection, pneumonia, lung cancer, and pneumothorax. A diagnostic algorithm for PE that includes CT is more cost effective than other diagnostic schemes. CT can correctly depict PE even in the presence of a coexisting pneumonia or preexisting lung disease.

The methods may prove particularly useful for certain types of patients in need of radiation diagnosis and treatment. These include, but are not limited to, pregnant patients who undergo lung and other CT scans, for children who need repeated chest CT examinations for metastatic surveys, for patients whose reproductive organs should be protected, for workers at work places with radiation hazards, and for patients who need cardiac catheterization and interventional cardiothoracic procedures. Undoubtedly, the specific dose reduction is a complex product that is affected by maternal body habitus, fetal gestational age, the position of the uterus, the orientation of the fetus, the quantity and concentration of the barium suspension consumed and its distribution through the GI tract. Furthermore, patients who are critically ill or may need caesarian section may not be the appropriate candidates for using oral barium.

### EXAMPLES

### Example 1

### The Attenuating Effect of Barium Sulfate as an Internal Shield to Protect the Fetus was Tested in a Phantom Simulation Experiment Involving Fetal Irradiation in Spiral Chest CT

An anthropomorphic Rando phantom with 2.5 cm thick tissue-equivalent contoured slabs was chosen as an in vitro model. The phantom had antero-posterior and side-to-side dimensions and a circumference of 27.5, 20.5, and 81 cm, respectively.

Two different LiF thermoluminescent detectors (TLDs), Harshaw and Landauer, 0.04 and 0.23 g/cm2 in weight, were chosen because (a) they can easily be positioned at various locations within the phantom; (b) a minimum dose of 0.10 mGy can be measured based on the variability of the signal from non-irradiated detectors despite background noise; (c) the measurement precision was better than 1.5% throughout the study; and (d) the energy response of the TLDs is known, having been investigated before at various beam qualities. The last characteristic was particularly important because the energy spectrum of the scattered radiation is not well known. The TLDs were calibrated in a 6 MV phantom beam with a calibrated ionization chamber. A correction factor of 1.6 was applied to the 6 MV TLD calibrations to account for the energy response of the detector in the CT beam.

For measurement of the attenuation of the internal scatter for a lung CT scan, two volumes of water, two concentrations of barium, 2% and 40%, and one thickness of lead shielding were used to perform a chest CT scan in an adult phantom. Two hollow acrylic slabs, 1 cm and 2 cm thick, were made to fit seamlessly between the phantom's slabs; the acrylic slabs had capacities of 315 and 630 cc, respectively. These two different volumes were chosen to compare their attenuation of the scattered radiation. The slabs were filled first with water as a control and then with 2% and 40% concentrations of barium sulfate, and were placed between the lowest point of the thorax and the upper abdomen of the phantom at the L1 level. 3 mm×3 mm square TLDs of two different thicknesses were housed in 15 round slots, 5 mm in diameter, in the phantom slab and placed first in the near field (z=0) corresponding to the L2 plane, immediately below the slabs, and the second at the far field (L5 level), at z=10 cm, caudal to z=0, to represent the approximate positions of the dome of the uterus in last and first trimesters of pregnancy respectively. To block the external source of radiation, a one-mm-thick lead sheet was wrapped around the lower portion of the phantom containing the TLDs.

In all, 17 experiments were performed. Eight were done with one-and two-cm-thick slabs containing water as control, and 8 others were conducted with slabs containing 2% or 40% barium in the near field and in the far field. For each experiment, a new set of TLDs was used. Experiment #17 was performed with a 1 mm thick lead sheet, placed between the thorax and the TLDs as the reference shield. A single-detector row Phillips spiral CT equipment was used. The exposure factors were 130 kVp, 230 mAs, 3 mm slice thickness, 1.7 pitch and 30 cm collimation from the lung apices to lung bases. These factors remained constant throughout the 17 experiments. In none of the experiments was a CT scout image acquired.

Attenuation measurements were made during a CT scan with 0.5 mm and 1.0 mm lead foils to obtain information on the energy of the scattered radiation which was needed for TLD calibration and to provide an effective broad beam attenuation coefficient.

An uncertainty analysis was performed because in converting the measured TLD signal to dose four different factors must be taken into account. These were calibration of TLDs at 6 MV, TLDs individual sensitivity, TLD energy exposure and background signal. This energy is consistent with finding that there was no difference in the dose measurements obtained from the two different thickness detectors in this study.

The control radiation dose measured in eight different experiments with water slabs at the near field was 3.06−4.14(3.60+0.54) mSvs, and for the far field it was 0.437−0.577(0.507+0.07) mSvs. The results of measurements of attenuation by two different thicknesses of barium sulfate relative to two thickness of water for 0.3 mm helical CT of two different TLD thicknesses are presented in Table I. Column two lists averaged measured doses in the homogeneous phantom for a typical CT scan. These data are given to provide some perspective on the magnitude of the scattered radiation that is preferably attenuated by barium sulfate.

TABLE I
Summary of attenuation measurements (A_{eff}) of barium sulfate relative to water, in the Rando phantom for 0.3 cm helical CT scan at 130 kVp and 230 mA.
+UZ,-16/49 A_{eff} = 1 − D_{slab}/D_{wat}

| | TLD - 0.04 g/cm² | | | | TLD - 023 g/cm² | | | |
|---|---|---|---|---|---|---|---|---|
| | 2% solution | | 40% solution | | 2% solution | | 40% solution | |
| Z* | 1 cm | 2 cm | 1 cm | 2 cm | 1 cm | 2 cm | 1 cm | 2 cm |
| 0 | 0.13 | 0.20 | 0.90 | 0.97 | 0.11 | 0.19 | 0.89 | 0.98 |
| 10 | 0.17 | 0.24 | 0.86 | 0.94 | 0.13 | 0.20 | 0.83 | 0.92 |

*Z is the distance in cm from the fluid filled slab to the TLD containing Rando slab

The effective attenuation, A_{eff}, given in Table I, is one minus the ratio of the dose measured with barium sulfate to the dose measured with an equivalent thickness of water replacing the barium sulfate, that is,
A_{eff}=1−A_{slab}/D_{tissue.} (1)

Different thickness detectors gave the same results within the range of estimated experimental uncertainties, and the percent attenuation was the same at z=0 and z=10 cm. With 0.04 g/cm² TLDs, the attenuations for a 2% concentration of barium sulfate suspension were 13% and 17% for a 1 cm and 2 cm slab thickness at the near and the far field, respectively. These values were 11% and 13% for TLDs 0.023 g/cm². For a 40% barium concentration, depending on the slab, the near- or far-field measurements, and the two different TLDs, the attenuation ranged between 86% and 98%. If we assume that the scattered radiation is attenuated by the barium suspension as an exponential function of barium concentration, then, we obtain the following relationship:
D_{slab}/Dₜᵢₛₛᵤₑ=exp(−μ_{eff}×concentration×t), (2)
where μ_{eff} is an effective linear attenuation coefficient and t is the thickness of the barium sulfate layer. Using data from the attenuation by the 2% and 40% concentrations, we obtained an effective linear attenuation coefficient μ_{eff}=5.9 cm⁻¹. Substituting equation (2) in equation (1), we obtain
   A_{eff}=1−exp(−μ_{eff}×concentration×t). (3)

With this equation, the A_{eff} of the suspension may be estimated for different concentrations and thicknesses of barium suspension. Table II projects A_{eff} for barium concentrations from 5 to 40% in 5% increments. As an example, for a 20% concentration we calculate attenuations of 69% and 90% for a 1 cm and a 2 cm thickness of barium sulfate, respectively, in the near and far fields.

TABLE II
The effective attenuation for various concentrations
of barium sulfate in slabs of different thickness

| % Concentration BaSO₄ | 1 cm slab | 2 cm slab |
|---|---|---|
| 5 | 0.26 | 0.45 |
| 10 | 0.45 | 0.69 |
| 15 | 0.59 | 0.83 |
| 20 | 0.69 | 0.91 |
| 25 | 0.77 | 0.95 |
| 30 | 0.83 | 0.97 |
| 35 | 0.87 | 0.98 |
| 40 | 0.91 | 0.99 |

Using the attenuation data for 2% barium the composite exponential attenuation profile is illustrated in FIG. 1 for one- and two-cm-thick slabs for barium concentrations of 2%-40% in the near and far fields. The one-mm lead sheet placed between the thorax and the abdomen attenuated 99% of the scattered photons. From the experiment with 0.5 mm and 1.0 mm lead foil we established an effective energy of 60 KcV for the scattered radiation.

Uncertainty Analysis. In the analysis of attenuation by barium sulfate, some of the uncertainties were eliminated because the ratio of the TLD signals was evaluated. However, in converting the measured TLD signal to dose, four factors were involved. For each factor, one standard deviation (s.d.) of uncertainty was:
σ(calibration at 6 MV)=±2%
σ(TLD individual sensitivity)=±1.5%
σ(TLD energy response)=±7%

$$\begin{matrix}σ ( background signal ) = \pm 1 \% at z = 0 cm \\ \pm 10 \% at z = 10 cm .\end{matrix}$$

Assuming that the individual uncertainties add in quadrature, the estimated uncertainties in the attenuation measurements at 1 s.d. or at the 68% confidence level are +8% and +13% at z=0 cm and 10 cm, respectively.

In the experiments with water slabs, the control radiation doses were 3.06-4.14 mSvs in near field, the presumed position of the dome of the uterus in the third trimester. The far field dose was 0.43-0.57 mSv simulating the radiation dose in the first trimester. Because, with up to four-slice MDCT, the radiation dose can increase by 30%-100%, the adjusted near-field radiation dose with this version of MDCT is preferably 4.68 mSvs to 8.28 mSv. These doses without any additional radiation from scout films or other imaging studies using ionizing radiation, are almost equal to or more than the 5 mSv limit set by the ICRP for the total 9-month occupational dose allowed to a pregnant worker and are 10-16 times greater than that allowed for the monthly radiation dose. The fetal gonadal dose in the third trimester, when the fetal head is engaged in the mother's pelvis and the fetal gonads, specifically the testes, are at the near field, is significantly higher.

The protective effect of 40% barium sulfate with TLDs of two different thicknesses was 90-98% in the near field and 86 to 92% in the far field. However, a 40% barium suspension need not be used in every patient. The extrapolated data offer the pregnant patient several choices of barium volume and concentration. Both 630 cc³ of 20% and 315 cc³ of 40% barium suspension can provide 91% attenuation. Even a 5-10% barium concentration can save the fetus 45%-69% of radiation exposure (FIG. 1). At a 30% concentration, the attenuation curves plateau irrespective of the volume of barium consumed or the position of TLDs in the near or the far field. Beyond 30%, further radiation savings is preferably only slight. Therefore, the effectiveness of 30% or more of barium suspension approaches the attenuating effect of a 1 mm-thick lead sheet. It is noteworthy that the attenuating effect of 40% barium suspension during the actual experiment for 1 cm and 2 cm slabs was the same as the values obtained by the extrapolation method (90%-98% vs. 91%-99%). This gives credence to the extrapolation technique and to the projected data provided in the present invention.

For the far field (z=10 cm), the presumed position of the uterus during the first trimester, the radiation dose was 0.437-0.577 mSv. Low as it may seem to be, this dose is delivered during the critical phase of organogenesis when the fetus/embryo is most sensitive to radiation. The barium shield attenuates the fetal dose in this critical period by 83%-94%.

The study in a phantom shows that a 30%-40% barium sulfate shield attenuates scattered photons almost as effectively as does a one mm lead shield. The method is not rigid and gives the patient and the radiologist latitude in the choice of volume and concentration of barium without substantially altering the attenuation effect. Above a 30% concentration, any further saving in radiation dose is only slight. This method is practical, and can safeguard the health of the fetus, in accordance with the ALARA philosophy. This method can be used for imaging studies involving various types of ionizing radiation.

The use of oral barium salt is safe, effective, and intuitively logical. It can potentially save 1-10 lifetime malignancies/10,000 pregnant patients who undergo chest CT examinations. Its use can alleviate the apprehension of acutely ill pregnant patients, their families and their physicians—practically at no cost.

It should be understood by one skilled in the art that the method can be adapted to allow a slight increase in the amount of scattered radiation in exchange for the use of less concentrated and more palatable barium preparations. A significant reduction in scattered radiation is achieved with a solution containing 20% barium, ranging between 75% for 1 cm thickness barium shield in near field and 95% for 2 cm thickness barium shield in far field.

The invention having been fully described, it is preferably apparent to one skilled in the art that changes and modifications can be made thereto without departing from the spirit and scope thereof.

What is claimed is:

1. A method of irradiating a patient comprising the steps of:
a) administering a radiation-attenuating agent within a first portion of a patient body cavity;
b) irradiating a second portion of the patient body cavity with a therapeutically effective source of radiation; wherein the second portion is different from the first portion; and wherein the administered radiation-attenuating agent is positioned to attenuate at least a portion of the radiation incident upon the first portion of the patient body cavity.

2. The method of irradiating a patient of claim 1, where the radiation-attenuating agent comprises a barium salt.

3. The method of irradiating a patient of claim 1, where the irradiation of the second portion of the body cavity is performed as part of a diagnostic imaging procedure.

4. The method of irradiating a patient of claim 3, where the diagnostic imaging procedure is a computed tomography scan or an X-ray.

5. The method of irradiating a patient of claim 3, where the diagnostic imaging procedure is a diagnostic procedure for detecting pulmonary embolism.

6. The method of irradiating a patient of claim 1, where the radiation is X-ray radiation.

7. The method of irradiating a patient of claim 1, where the patient is pregnant or believed to be pregnant.

8. The method of irradiating a patient of claim 1, where the first portion of the patient body cavity is at least a portion of the alimentary canal of the patient.

9. The method of irradiating a patient of claim 1, where the second portion of the patient body cavity is at least a portion of the thorax of the patient.

10. The method of irradiating a patient of claim 1, where the radiation-attenuating agent is administered orally.

11. The method of irradiating a patient of claim 1, where the radiation-attenuating agent is administered orally as a solution comprising barium.

12. The method of irradiating a patient of claim 1, further comprising the steps of:
a) detecting radiation passing through the second portion of the patient body cavity; and
b) providing a diagnostic image showing at least the second portion of the patient body cavity.

13. A method of diagnostic imaging comprising the steps of:
a) administering a radiation-attenuating agent to the gastrointestinal tract;
b) irradiating a portion of the patient body cavity with a diagnostic dose of X-ray radiation, where the portion of the patient body cavity is in addition to the gastrointestinal tract;
c) imaging the irradiated portion of the patient body.

14. The method of diagnostic imaging of claim 13, where the radiation-attenuating agent comprises barium.

15. The method of diagnostic imaging of claim 13, further comprising the steps of:
a) detecting radiation passing through a third portion of the patient body cavity; and
b) providing a diagnostic image showing at least the third portion of the patient body cavity.

16. A method of detecting a clinical condition comprising the steps of:
a) administering a radiation-attenuating agent comprising barium within a first portion of a patient body cavity;
b) performing a diagnostic imaging procedure of a second portion of the patient body cavity with a diagnostic dose of X-ray radiation; wherein the second portion is different from the first portion; and wherein the administered radiation-attenuating agent is positioned to attenuate at least a portion of the radiation incident upon a gonad or the uterus of the patient during the diagnostic imaging procedure.

17. The method of detecting a clinical condition of claim 16, further comprising the step of collecting a diagnostic image of the second portion of the body cavity, where the clinical condition is a pulmonary embolism, and the diagnostic imaging procedure is a computed tomography scan.

18. A method comprising the steps of:
a) administering a barium salt solution into the gastrointestinal tract of a woman bearing a fetus; and
b) irradiating the body of the woman at a site other than her uterus.
